(19) United States
(12) Patent Application Publication
(10) Pub. No.: US 2003/0125304 A1
(43) Pub. Date: Jul. 3, 2003

(54) Substituted amino ketone compounds

(76) Inventors: Hans-Ulrich Demuth, Halle/Saale (DE); Ulrich Heiser, Halle/Saale (DE); Torsten Hoffmann, Halle/Saale (DE); Andre Niestroj, Halle/Saale (DE)

Correspondence Address: Brown Rudnick Berlack Israels LLP, 18th Floor, Box IP, One Financial Center, Boston, MA, 02111, US

(21) Appl. No.: 10/287,300

(22) Filed: Nov. 4, 2002

Related U.S. Application Data
(60) Provisional application No. 60/340,182, filed on Dec. 14, 2001.

(30) Foreign Application Priority Data
Nov. 11, 2001 (DE) DE 101 54 689.0

(51) Int. Cl. C07F 9/28; A61K 31/675; A61K 31/4709; A61K 31/4439; C 07D 4 1/02; C 07D 4 3/02

(52) U.S. Cl. 514/80; 514/307; 514/339; 514/381; 546/23; 546/148; 546/268.1; 548/252

(57) ABSTRACT

The present invention relates to compounds of the general formula I

B—(CH—R¹)ₙ—C(═X²)—D (I)

and pharmaceutically acceptable salts thereof including stereoisomers, to the use of the compounds for the treatment of impaired glucose tolerance, glucosuria, hyperlipidaemia, metabolic acidosis, diabetes mellitus, diabetic neuropathy and nephropathy and of sequelae caused by diabetes mellitus in mammals.

### FIELD OF THE INVENTION

The present invention relates to substituted amino ketone compounds and salts thereof, hereinafter referred to as amino ketones, and to the use of the compounds for the preparation of a medicament for the in vivo inhibition of DP IV and/or DP IV-like enzymes.

The invention relates especially to the use of the compounds for the preparation of a medicament for the treatment of impaired glucose tolerance, glucosuria, hyperlipidaemia, metabolic acidosis, diabetes mellitus, diabetic neuropathy and nephropathy and of sequelae caused by diabetes mellitus in mammals, for the treatment of metabolism-related hypertension and of cardiovascular sequelae caused by hypertension in mammals, for the prophylaxis or treatment of skin diseases and diseases of the mucosae, autoimmune diseases and inflammatory conditions, and for the treatment of psychosomatic, neuropsychiatric and depressive illnesses, such as anxiety, depression, sleep disorders, chronic fatigue, schizophrenia, epilepsy, nutritional disorders, spasm and chronic pain.

### BACKGROUND OF THE INVENTION

Dipeptidyl peptidase IV (DP IV) is a post-proline (to a lesser extent post-alanine, post-serine or post-glycine) cleaving serine protease found in various tissues of the body including kidney, liver, and intestine, where it removes dipeptides from the N-terminus of biologically active peptides with a high specificity when proline or alanine form the residues that are adjacent to the N-terminal amino acid in their sequence.

Among the rare group of proline-specific proteases, DP IV was originally believed to be the only membrane-bound enzyme specific for proline as the penultimate residue at the amino-terminus of the polypeptide chain. However, other molecules, even structurally non-homologous with the DP IV but bearing corresponding enzyme activity, have been identified. DP IV-like enzymes, which are identified so far, are e.g. fibroblast activation protein α, dipeptidyl peptidase IV β, dipeptidyl aminopeptidase-like protein, N-acetylated α-linked acidic dipeptidase, quiescent cell proline dipeptidase, dipeptidyl peptidase II, attractin and dipeptidyl peptidase IV related protein (DPP 8), DPL1 (DPX, DP6) and DPL2, and are described in the review articles by Sedo & Malik (Sedo & Malik, Dipeptidyl peptidase IV-like molecules: homologous proteins or homologous activities? Biochimica et Biophysica Acta 2001, 36506: 1-10) and Abbott & Gorrell (Abbott, C. A. & Gorrell, M. D., The family of CD26/DP IV and related ectopeptidases. In: Langner & Ansorge (ed.), Ectopeptidases. Kluwer Academic/Plenum Publishers, New York, 2002, pp. 171-195).

Further DP IV-like enzymes are disclosed in WO 01/19866, WO 02/34900 and WO02/31134. WO 01/19866 discloses novel human dipeptidyl aminopeptidase 8 (DPP8) with structural and functional similarities to DP IV and fibroblast activation protein (FAP). WO 02/34900 discloses a novel dipeptidyl peptidase 9 (DPP9) with significant homology to the amino acid sequences of DP IV and DPP8. WO 02/31134 discloses three DP TV-like enzymes, DPRP1, DPRP2 and DPRP3. Sequence analysis revealed that DPRP1 is identical to DPP8 as disclosed in WO 01/19866, that DPRP2 is identical to DPP9 and that DPRP3 is identical to KIAAA1492 as disclosed in WO 02/04610.

Likewise, it has been found that DP IV is responsible for inactivating glucagon-like peptide-1 (GLP-1) and glucose-dependent insulinotropic peptide also known as gastric-inhibitory peptide (GIP). Since GLP-1 is a major stimulator of pancreatic insulin secretion and has direct beneficial effects on glucose disposal, in WO 97/40832 and U.S. Pat. No. 6,303,661 inhibition of DP IV and DP IV-like enzyme activity was shown to represent an attractive approach for treating non-insulin-dependent diabetes mellitus (NIDDM).

The reduction of such DP IV and DP IV-like enzyme activity for cleaving such substrates in vivo can serve to suppress undesirable enzyme activity effectively both under laboratory conditions and in pathological conditions of mammals. For example, Diabetes mellitus type II (also diabetes of old age) is based upon reduced insulin secretion or disturbances in receptor function which are founded inter alia upon proteolytically determined abnormalities in the concentration of the incretins.

Hyperglycaemia and its associated causes and sequelae (also Diabetes mellitus) are treated according to the current state of the art by administering insulin (for example material isolated from bovine pancreas or also material obtained by genetic engineering) to those affected, in various forms of administration. All of the previously known methods and also more modem methods are characterised by high expenditure on materials, high costs and often by crucial impairment of the patient's life quality. The classical method (daily i.v. insulin injection, customary since the thirties) treats the acute symptoms of the disease but leads, after prolonged use, to inter alia severe vascular changes (arteriosclerosis) and nerve damage.

It is known that DPIV-inhibitors may be useful for the treatment of impaired glucose tolerance and diabetes mellitus (International Patent Application, Publication Number WO 99/61431, Pederson R A et al, Diabetes. August; 1998 47(8):1253-8 and Pauly R P et al, Metabolism March; 1999 48(3):385-9). In particular WO 99/61431 discloses DPIV-Inhibitors comprising an amino acid residue and a thiazoleidine or pyrrolidine group, and salts thereof, especially L-threo-isoleucyl thiazoleidine, L-allo-isoleucyl thiazoldine, L-threo-isoleucyl pyrrolidine, L-allo-isoleucyl thiazoldine, L-allo-isoleucyl pyrrolidine, and salts thereof

Further examples of low molecular weight dipeptidyl peptidase IV inhibitors are agents such as tetrahydroisoquinolin-3-carboxamide derivatives, N-substituted 2-cyanopyrroles and -pyrrolidines, N-(N′-substituted glycyl)-2-cyanopyrrolidines, N-(substituted glycyl)-thiazoldines, N-(substituted glycyl)-4-cyanothiazoldines, amino-acyl-borono-prolyl-inhibitors and cyclopropyl-fused pyrrolidines. Inhibitors of dipeptidyl peptidase IV are described in U.S. Pat. No. 6,011,155; U.S. Pat. No. 6,107,317; U.S. Pat. No. 6,110,949; U.S. Pat. No. 6,124,305; U.S. Pat. No. 6,172,081; WO 99/61431, WO 99/67278, WO 99/67279, DE 198 34 591, WO 97/40832, DE 196 16 486 C 2, WO 98/19998, WO 00/07617, WO 99/38501, WO 99/46272, WO 99/38501, WO 01/68603, WO 01/40180, WO 01/81337, WO 01/81304, WO 01/55105, WO 02/02560 and WO 02/14271, WO 02/076450, WO 02/051836, EP 02290755.4 and WO 02/38541, the teachings of which are herein incorporated by reference in their entirety concerning these inhibitors, their uses, definition and their production.

More recently, the installation of subcutaneous depot implants (the insulin is released in metered amounts, and daily injections are unnecessary) and the implantation (transplantation) of intact Langerhans cells into the dysfunctional pancreas gland or other organs and tissues have been proposed. Such transplantation is complicated from a technical point of view. It furthermore represents risky surgical intervention in the recipient and, in the case of cell transplantation, also requires methods of suppressing or by-passing the immune system.

The problem of the invention is therefore to provide new compounds for the treatment of, for example, impaired glucose tolerance, glucosuria, hyperlipidaemia, metabolic acidosis, diabetes mellitus, diabetic neuropathy and nephropathy and of sequelae caused by diabetes mellitus in mammals, metabolism-related hypertension and cardiovascular sequelae caused by hypertension in mammals, for the prophylaxis or treatment of skin diseases and diseases of the mucosae, autoimmune diseases and inflammatory conditions, and for the treatment of psychosomatic, neuropsychicatric and depressive illnesses, such as anxiety, depression, sleep disorders, chronic fatigue, schizophrenia, epilepsy, nutritional disorders, spasm and chronic pain, and a simple method for the treatment of those diseases.

### SUMMARY OF THE INVENTION

This invention comprises compounds of the general formula I

B—(CH—R¹)ₙ—C(═X²)—D (I)

wherein

n is 0 or 1,

R¹ stands for H, C₁-C₉ branched or straight chain alkyl, n-butan-2-yl, n-prop-2-yl or isobutyl, C₂-C₉ branched or straight chain alkenyl, C₃-C₈ cycloalkyl, C₅-C₇ cycloalkenyl, aryl, heteroaryl or a side chain of a natural amino acid or derivatives thereof,

X² stands for O, NR⁶, N⁺(R⁷)₂, or S,

B is selected from the following groups:

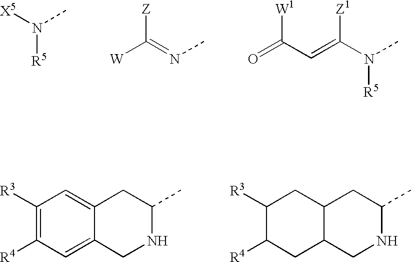

where X⁵ is H or an acyl or oxycarbonyl group including amino acids,

R⁵ is H, C₁-C₉ branched or straight chain alkyl, C₂-C₉ branched or straight chain alkenyl, C₃-C₈ cycloalkyl, C₅-C₇ cycloalkenyl, aryl, heteroaryl or a side chain of a natural amino acid or mimetics thereof, or a group of the formula —(CH)ₘ—NH—C₅H₃N—Y where m is an integer of 2-4, —C₅H₃N—Y is a divalent pyridyl moiety and Y is a hydrogen atom, a halogen atom, a nitro group or a cyano group,

Z is selected from H, pyridyl or optionally substituted phenyl, optionally substituted alkyl groups, alkoxy groups, halogens, nitro, cyano and carboxy groups,

W is selected from H, pyridyl or optionally substituted phenyl, optionally substituted alkyl groups, alkoxy groups, halogens, nitro, cyano and carboxy groups,

W¹ is H or optionally substituted alkyl, alkoxy or optionally substituted phenyl, and

Z¹ is H, or optionally substituted alkyl,

R³ and R⁴ are independently H, hydroxy, alkyl, alkoxy, aralkoxy, nitro, cyano or halogen,

D is an optionally substituted compound of the formula

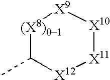

which can be saturated, or can have one, two or three double bonds, wherein

X⁸ to X¹¹ are independently CH, N, N⁺(R⁷), or CR⁸, if unsaturated, or

X⁸ to X¹¹ are independently CH₂, NH, NH⁺(R⁷), O, or S if saturated,

X¹² is CHA, NA, CH₂, NH, NH⁺(R⁷), or CHR⁸, if saturated or

X¹² is CA, NA⁺, CH, N, N⁺(R⁷), or CR⁸, if unsaturated and

A is H or an isoster of a carboxylic acid, PO₃R⁵R⁶, a tetrazole, an amide, an ester or an acid anhydride,

R⁶, R⁷ R⁸and R⁹ are independently selected from H, optionally substituted C₁-C₉ branched or straight chain alkyl, or optionally substituted C₂-C₉ branched or straight chain alkenyl, or optionally substituted C₃-C₈ cycloalkyl, or an optionally substituted C₅-C₇ cycloalkenyl, or an optionally substituted aryl residue.

### DETAILED DESCRIPTION OF THE INVENTION

That problem is solved according to the invention by providing compounds of the general formula I including all stereoisomers:

B—(CH—R¹),—C(═X²)—D (I)

wherein

n is 0 or 1,

R¹ stands for H, C₁-C₉ branched or straight chain alkyl, preferably H, n-butan-2-yl, n-prop-2-yl or isobutyl, C₂-C₉ branched or straight chain alkenyl, C₃-C₈ cycloalkyl, preferably cyclohexyl, C₅-C₇ cycloalkenyl, aryl, heteroaryl or a side chain of a natural amino acid or mimetics thereof,

X² stands for O, NR⁶, N⁺(R⁷)₂, or S,

B is selected from the following groups:

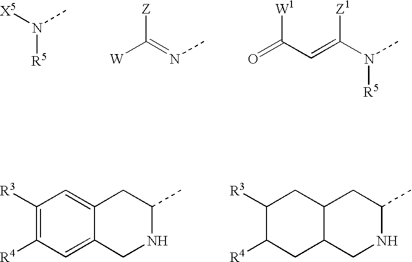

where X⁵ is H or an acyl or oxycarbonyl group including amino acids,

R⁵is H, C₁-C₉ branched or straight chain alkyl, preferably H, n-butan-2-yl, n-prop-2-yl or isobutyl, C₂-C₉ branched or straight chain alkenyl, C₃-C₈ cycloalkyl, preferably cyclohexyl, 3-hydroxyadamant-1-yl, C₅-C₇ cycloalkenyl, aryl, heteroaryl or a side chain of a natural amino acid or derivatives thereof, or a group of the formula —(CH)ₘ—NH—C₅H₃N—Y where m is an integer of 2-4, —C₅H₃N—Y is a divalent pyridyl moiety and Y is a hydrogen atom, a halogen atom, a nitro group or a cyano group,

R⁶, R⁷ R⁸ and R⁹ are independently selected from H, optionally substituted C₁-C₉ branched or straight chain alkyl, preferably an optionally substituted C₂-C₅ branched or straight chain alkyl; or optionally substituted C₂-C₉ branched or straight chain alkenyl, preferably an C₂-C₅ branched or straight chain alkenyl; or optionally substituted C₃-C₈ cycloalkyl, preferably an optionally substituted C₄-C₇ cycloalkyl; or an optionally substituted C₅-C₇ cycloalkenyl, or an optionally substituted aryl residue,

Z is selected from H, pyridyl or optionally substituted phenyl, optionally substituted alkyl groups, alkoxy groups, halogens, nitro, cyano and carboxy groups,

W is selected from H, pyridyl or optionally substituted phenyl, optionally substituted alkyl groups, alkoxy groups, halogens, nitro, cyano and carboxy groups,

W¹ is H or optionally substituted alkyl, alkoxy or optionally substituted phenyl, and Z¹ is H, or optionally substituted alkyl,

R³ and R⁴ are independently H, hydroxy, alkyl, alkoxy, aralkoxy, nitro, cyano or halogen,

D is an optionally substituted compound of the formula

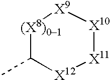

which can be saturated, or can have one, two or three double bonds, wherein

X⁸ to X¹¹ are independently CH, N, N⁺(R⁷), or CR⁸, if unsaturated, or

X⁸ to X¹¹ are independently CH₂, NH, NH⁺(R⁷), O, or S if saturated,

X¹² is CHA, NA, CH₂, NH, NH⁺(R⁷), or CHR⁸, if saturated or

X¹² is CA, NA⁺, CH, N, N⁺(R⁷) or CR⁸, if unsaturated and

A is H or an isoster of a carboxylic acid such as CN, SO₃H, CONOH, PO₃R⁵R⁶, a tetrazole, an amide, an ester or an acid anhydride.

Throughout the application, D contains preferably at most two, further preferred at most one hetero atom in the ring.

According to preferred embodiments of the present invention, D stands for optionally substituted C₄-C₇ cycloalkyl, preferably C₄-C₆ cycloalkyl, optionally substituted C₄-C₇ cycloalkenyl, or optionally substituted (hetero)cycloalkyl of the formulae

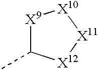

wherein the residues are as defined above, or

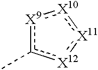

that is, a five-membered ring containing one or two double bonds in the ring, wherein the residues are as defined above, or

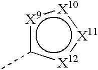

wherein the residues are as defined above, or

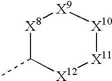

wherein the residues are as defined above, or

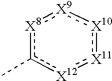

that is a six-membered ring containing one or two double bonds in the ring,

wherein the residues are as defined above, or

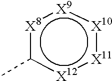

wherein the residues are as defined above.

According to a preferred embodiment, B has the following formula:

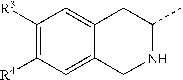

wherein the residues are as defined above.

According to another preferred embodiment, B has the following formula:

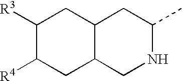

wherein the residues are as defined above.

Throughout the description and the claims the expression “optionally substituted” preferably means any alkyl, acyl, aryl, heteroaryl, carbonyl, carboxyl, halogenyl moiety.

“Acyl” can denote a C1-20 acyl residue, preferably a C1-8 acyl residue and especially preferred a C1-4 acyl residue; “cycloalkyl” can denote a C3-12 cycloalkyl residue, preferably a C4, C5 or C6 cycloalkyl residue; and “carbocyclic” can denote a C3-12 carbocyclic residue, preferably a C4, C5 or C6 carbocyclic residue. “Heteroaryl” is defined as an aryl residue, wherein 1 to 4, and more preferably 1, 2 or 3 ring atoms are replaced by heteroatoms like N, S or O. “Heterocyclic” is defined as a cycloalkyl residue, wherein 1, 2 or 3 ring atoms are replaced by heteroatoms like N, S or O. “Peptides” are selected from dipeptides to decapeptides, preferred are dipeptides, tripeptides, tetrapeptides and pentapeptides. The amino acids for the formation of the “peptides” can be selected from those listed below.

“Chronic” as used in reference to therapeutic administration of drug shall mean administration on at least about 5 days in any thirty day period. In addition chronic administration comprises extending for at least about 2 months. In some embodiments this extends to at least about six months, and further at least about one year.

According to a preferred embodiment the acyl groups are C1-C6-acyl groups.

According to a further preferred embodiment the alk(yl) groups are C1-C6-alk(yl) groups, which may be branched or unbranched.

According to a further preferred embodiment the alkoxy groups are C1-C6-alkoxy groups.

According to a further preferred embodiment the aryl residues are C5-C12 aryl residues that have optionally one, two or three fused rings having, e.g. 3, 4 or 5 additional C-atoms each.

According to a further preferred embodiment the cycloalkyl residues (carbocycles) are C3-C8-cycloalkyl residues.

According to a further preferred embodiment the heteroaryl residues are C4-C 11 aryl residues that have optionally one, two or three fused rings having, e.g. 3, 4 or 5 additional C-atoms each and, in at least one ring, additionally from 1 to 4 preferably 1 or 2 hetero atoms, such as O, N and/or S.

According to a further preferred embodiment peptide residues are corresponding residues containing from 2 to 50 amino acids.

According to a further preferred embodiment the heterocyclic residues are C2-C7-cycloalkyl radicals that additionally have from 1 to 4, preferably 1 or 2 hetero atoms, such as O, N and/or S.

According to a further preferred embodiment the carboxy groups are C1- C6 carboxy groups, which may be branched or unbranched.

According to a further preferred embodiment the oxycarbonyl groups are groups of the formula —O—(CH₂)₁₋₆COOH.

The amino acids can be any natural or synthetic amino acid, preferably natural alpha amino acids.

Examples of amino acids which can be used in the present invention are L and D-amino acids, N-methyl-amino-acids; allo- and threo-forms of Ile and Thr, which can, e.g. be α-, β- or ω-amino acids, whereof α-amino acids are preferred.

Examples of amino acids are:

aspartic acid (Asp), glutamic acid (Glu), arginine (Arg), lysine (Lys), histidine (His), glycine (Gly), serine (Ser) and cysteine (Cys), threonine (Thr), asparagine (Asn), glutamine (Gln), tyrosine (Tyr), alanine (Ala), proline (Pro), valine (Val), isoleucine (Ile), leucine (Leu), methionine (Met), phenylalanine (Phe), tryptophan (Trp), hydroxyproline (Hyp), beta-alanine (beta-Ala), 2-amino octanoic acid (Aoa), azetidine-(2)-carboxylic acid (Ace), pipecolic acid (Pip), 3-amino propionic, 4-amino butyric and so forth, alpha-aminoisobutyric acid (Aib), sarcosine (Sar), ornithine (Orn), citrulline (Cit), homoarginine (Har), t-butylalanine (t-butyl-Ala), t-butylglycine (t-butyl-Gly), N-methylisoleucine (N-MeIle), phenylglycine (Phg), cyclohexylalanine (Cha), norleucine (Nle), cysteic acid (Cya) and methionine sulfoxide (MSO), Acetyl-Lys, modified amino acids such as phosphoryl-serine (Ser(P)), benzyl-serine (Ser(Bzl)) and phosphoryl-tyrosine (Tyr(P)), 2-aminobutyric acid (Abu), aminoethylcysteine (AECys), carboxyrnethylcysteine (Cmc), dehydroalanine (Dha), dehydroamino-2-butyric acid (Dhb), carboxyglutaminic acid (Gla), homoserine (Hse), hydroxylysine (Hyl), cis-hydroxyproline (cisHyp), trans-hydroxyproline (transHyp), isovaline (Iva), pyroglutamic acid (Pyr), norvaline (Nva), 2-aminobenzoic acid (2-Abz), 3- aminobenzoic acid (3-Abz), 4-aminobenzoic acid (4-Abz), 4-(aminomethyl)benzoic acid (Amb), 4-(aminomethyl)cyclohexanecarboxylic acid (4-Amc), Penicillamine (Pen), 2-Amino-4-cyanobutyric acid (Cba), cycloalkane-carboxylic aicds.

Examples of \overline{ω}-amino acids are e.g.: 5-Ara (aminoraleric acid), 6-Ahx (aminohexanoic acid), 8-Aoc (aminooctanoic aicd), 9-Anc (aminovanoic aicd), 10-Adc (aminodecanoic acid), 11 -Aun (aminoundecanoic acid), 12-Ado (aminododecanoic acid).

Further amino acids are: indanylglycine (Igl), indoline-2-carboxylic acid (Idc), octahydroindole-2-carboxylic acid (Oic), diaminopropionic acid (Dpr), diaminobutyric acid (Dbu), naphtylalanine (1-Nal), (2-Nal), 4-aminophenylalanin (Phe(4-NH₂)), 4-benzoylphenylalanine (Bpa), diphenylalanine (Dip), 4-bromophenylalanine (Phe(4-Br)), 2-chlorophenylalanine (Phe(2-Cl)), 3-chlorophenylalanine (Phe(3-Cl)), 4-chlorophenylalanine (Phe(4-Cl)), 3,4-chlorophenylalanine (Phe (3,4-Cl₂)), 3- fluorophenylalanine (Phe(3-F)), 4-fluorophenylalanine (Phe(4-F)), 3,4- fluorophenylalanine (Phe(3,4-F₂)), pentafluorophenylalanine (Phe(F₅)), 4-guanidinophenylalanine (Phe(4-guanidino)), homophenylalanine (hPhe), 3 jodophenylalanine (Phe(3-J)), 4 jodophenylalanine (Phe(4-J)), 4-methylphenylalanine (Phe(4-Me)), 4-nitrophenylalanine (Phe-4-NO₂)), biphenylalanine (Bip), 4-phosphonomehtylphenylalanine (Pmp), cyclohexyglycine (Ghg), 3-pyridinylalanine (3-Pal), 4-pyridinylalanine (4-Pal), 3,4-dehydroproline (A-Pro), 4-ketoproline (Pro(4-keto)), thioproline (Thz), isonipecotic acid (Inp), 1,2,3,4,-tetrahydroisoquinolin-3-carboxylic acid (Tic), propargylglycine (Pra), 6-hydroxynorleucine (NU(6-OH)), homotyrosine (hTyr), 3-jodotyrosine (Tyr(3-J)), 3,5-dijodotyrosine (Tyr(3,5-J₂)), d-methyl-tyrosine (Tyr(Me)), 3-NO₂-tyrosine (Tyr(3-NO₂)), phosphotyrosine (Tyr(PO₃H₂)), alkylglycine, 1-aminoindane-1-carboxy acid, 2-aminoindane-2-carboxy acid (Aic), 4-amino-methylpyrrol-2-carboxylic acid (Py), 4-amino-pyrrolidine-2-carboxylic acid (Abpc), 2-aminotetraline-2-carboxylic acid (Atc), diaminoacetic acid (Gly(NH₂)), diaminobutyric acid (Dab), 1,3-dihydro-2H-isoinole-carboxylic acid (Disc), homocylcohexylalanin (hCha), homophenylalanin (hP he oder Hof), trans-3-phenyl-azetidine-2-carboxylic acid, 4-phenyl-pyrrolidine-2-carboxylic acid, 5-phenyl0pyrrolidine-2-carboxylic acid, 3-pyridylalanine (3-Pya), 4-pyridylalanine (4-Pya), styrylalanine, tetrahydroisoquinoline-1-carboxylic acid (Tiq), 1,2,3,4-tetrahydronorharmane-3-carboxylic acid (Tpi), β-(2-thienyl)-alanine (Tha).

Side chains of amino acids are known to people skilled in the art: an amino acid has a backbone containing an amino and a carboxy group. Substituents of the backbone are called side chains.

Such side chains are for instance, but not restricted to, homoserine addition, pyroglutamic acid addition, disulphide bond formation, deamidation of asparagine or glutamine residues, methylation, t-butylation, t-butyloxycarbonylation, 4-methylbenzylation, thioanysilation, thiocresylation, benzyloxymethylation, 4-nitrophenylation, benzyloxycarbonylation, 2-nitrobencoylation, 2-nitrosulphenylation, 4-toluenesulphonylation, pentafluorophenylation, diphenylmethylation, 2-chlorobenzyloxycarbonylation, 2,4,5-trichlorophenylation, 2-bromobenzyloxycarbonylation, 9-fluorenylmethyloxycarbonylation, triphenylmethylation, 2,2,5,7,8,-pentamethylchroman-6-sulphonylation, hydroxylation, oxidation of methionine, formylation, acetylation, anisylation, benzylation, benzoylation, trifluoroacetylation, carboxylation of aspartic acid or glutamic acid, phosphorylation, sulphation, cysteinylation, glycolysation with pentoses, deoxyhexoses, hexosamines, hexoses or N-acetylhexosamines, famesylation, myristolysation, biotinylation, palmitoylation, stearoylation, geranylgeranylation, glutathionylation, 5′-adenosylation, ADP-ribosylation, modification with N-glycolylneuraminic acid, N-acetylneuraminic acid, pyridoxal phosphate, lipoic acid, 4′-phosphopantetheine, or N-hydroxysuccinimide.

Peptide mimetics per se are known to a person skilled in the art. They are preferably defined as compounds which have a secondary structure like a peptide and optionally further structural characteristics; their mode of action is largely similar or identical to the mode of action of the native peptide; however, their activity (e.g. as an antagonist or inhibitor) can be modified as compared with the native peptide, especially vis a vis receptors or enzymes. Moreover, they can imitate the effect of the native peptide (agonist). Examples of peptide mimetics are scaffold mimetics, non-peptidic mimetics, peptoides, peptide nucleic acids, oligopyrrolinones, vinylogpeptides and oligocarbamates. For the definitions of these peptide mimetics see Lexikon der Chemie, Spektrum Akademischer Verlag Heidelberg, Berlin, 1999.

The aim for using these mimetic structurs is increasing the activity, increasing the selectivity to decrease side effects, protect the compound (drug) against enzymatical degradation for prolongation of the effect.

Further peptide mimetics are defined in J. Gante, Angew. Chemie, 1994, 106, 1780-1802; V. J. Hruby et al., Biopolymers, 1997, 219-266; D. Nöteberg et al., 2000, 43, 1705-1713.

Upon—preferably oral—administration of those compounds to a mammal, the endogenous (or additionally exogenously administered) insulinotropic peptides GIP₁₋₄₂ and GLP-1₇₋₃₆ (or GLP-1₇₋₃₇ or analogues thereof), for example, are broken down to a lesser degree by DP IV or DP IV-like enzymes. The compounds of the present invention lower or inhibit the activity of DP IV or DP IV-like enzymes at least by about 10, preferably about 50, more preferably about 75, 90 or 100% and prolong the half live of their substrates in a mammal by at least about 2fold, preferably about 3fold, more preferably about 4fold, 5fold or higher relative to the absence of the compound and hence the reduction in the concentration of those peptide hormones and their analogues is reduced or delayed. The invention is based, therefore, on the finding that a reduction of the DP IV or DP IV-like enzyme activity in the bloodstream results in influencing of the blood sugar level. The compounds of the present invention are therefore useful for the treatment of impaired glucose tolerance, glucosuria, hyperlipidaemia, metabolic acidosis, diabetes mellitus, diabetic neuropathy and nephropathy and of sequelae caused by diabetes mellitus in mammals.

Besides the insulinotropic peptides GIP₁₋₄₂ and GLP-1₇₋₃₆ (or GLP1₇₋₃₇ or analogues thereof), the compounds of the present invention lower or inhibit the degradation of other substrates of DP IV or DP IV-like enzymes and are therefore useful for the treatment of metabolism-related hypertension and of cardiovascular sequelae caused by hypertension in mammals, for the prophylaxis or treatment of skin diseases and diseases of the mucosae, autoimmune diseases and inflammatory conditions, and for the treatment of psychosomatic, neuropsychiatric and depressive illnesses, such as anxiety, depression, sleep disorders, chronic fatigue, schizophrenia, epilepsy, nutritional disorders, spasm and chronic pain.

Currently known substrates of DP IV are

Xaa-Pro peptides

Tyr-melanostatin

Endomorphin-2

Enterostatin

β-Casomorphin

Trypsinogen pro-peptide

Bradykinin

Substance P

Corticotropin-like intermediate lobe peptide

Gastrin-releasing peptide

Neuropeptide Y

Peptide YY

Aprotinin

RANTES

GCP-2

SDF-1α

SDF-1β

MDC

MCP-1

MCP-2

MCP-3

Eotaxin

IP-10

Insulin-like growth factor-I

Pro-colipase

Interleukin-2

Interleukin-1β

α₁-Microglobulin

Prolactin

Trypsinogen

Chorionic gonadotropin

Xaa-Ala peptides

PHM

GRH-(1-29)

GRH-(1-44)

GLP-1

GLP-2

Gastric inhibitory peptide

Orexin B

Xaa-Ser peptides

Orexin A

The oral administration of the high-affinity, low-molecular-weight enzyme inhibitors of the invention is a more cost-effective alternative, for example, to invasive surgical techniques in the treatment of pathological symptoms. By chemical design of stability, transport and clearance properties their mode of action can be modified and matched to individual characteristics.

The salts of the compounds of the invention may, if they have basic properties, be in the form of inorganic or organic salts.

The compounds of the present invention can be converted into and used as acid addition salts, especially pharmaceutically acceptable acid addition salts. The pharmaceutically acceptable salt generally takes a form in which a basic side chain is protonated with an inorganic or organic acid. Representative organic or inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toulenesulfonic, cyclohexanesulfamic, salicylic, saccharinic or trifluoroacetic acid. All pharmaceutically acceptable acid addition salt forms of the compounds of the present invention are intended to be embraced by the scope of this invention.

In view of the close relationship between the free compounds and the compounds in the form of their salts, whenever a compound is referred to in this context, a corresponding salt is also intended, provided such is possible or appropriate under the circumstances.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms of the compounds may exist as polymorphs and as such are intended to be included in the present invention. In addition, some of the compounds may form solvates with water (i.e. hydrates) or common organic solvents, and such solvates are also intended to be encompassed within the scope of this invention.

The compounds, including their salts, can also be obtained in the form of their hydrates, or include other solvents used for their crystallization.

The invention accordingly relates to inhibitors of dipeptidyl peptidase TV (DPIV) and DP IV-like enzyme activity and to their use for lowering the blood sugar level below the glucose concentration characteristic of hyperglycaemia in the serum of a mammal. The invention relates especially to the use of the compounds of the invention for modulating DP IV and DP IV-like enzyme activity in order to prevent or alleviate pathological metabolic abnormalities of mammals, such as, for example, impaired glucose tolerance, glucosuria, hyperlipidaemia, metabolic acidosis, diabetes mellitus, diabetic neuropathy and nephropathy, and sequelae caused by diabetes mellitus in mammals. The invention further relates to the use of the compounds of the invention for modulating DP TV and DP IV-like enzyme activity in order to prevent or alleviate neurodegenerative diseases and high blood pressure. In the case of chronic administration of the compounds of the invention, the invention relates to the improvement of signal action at the cells of the islets of Langerhans and of insulin sensitivity in the peripheral tissue in the postprandial phase.

The invention further relates to the use of the compounds of the invention for the chronic treatment of chronic metabolic diseases in humans; for the chronic treatment of chronically impaired glucose tolerance, chronic glucosuria, chronic hyperlipidaemia, chronic metabolic acidosis, chronic diabetes mellitus, chronic diabetic neuropathy and nephropathy and of chronic sequelae caused by diabetes mellitus, chronic neurodegenerative diseases and chronic disturbance of signal action at the cells of the islets of Langerhans and chronic insulin sensitivity in the peripheral tissue in the postprandial phase of mammals; for the chronic treatment of chronic metabolism-related hypertension and of chronic cardiovascular sequelae caused by hypertension in mammals; for the chronic treatment of chronic psychosomatic, chronic neuropsychiatric and depressive illnesses, such as chronic anxiety, chronic depression, chronic sleep disorders, chronic fatigue, chronic schizophrenia, chronic epilepsy, chronic nutritional disorders, spasm and chronic pain.

The compounds of the present invention may be used in the form prodrugs. According to the invention, these prodrugs can be used as inhibitors of DP IV and DP IV-like enzymes and it is possible to define the site of their action, the time of onset of their action and the duration of action precisely.

Upon administration, such prodrugs are cleaved, for example by suitable enzymes, and the active inhibitors are released. The active inhibitors can be released both by chemical and enzymatic mechanisms. For example, esterases, proteases and peptidases serve to release the active inhibitors from the prodrugs according to the invention. Such esterases, proteases, etc. are disclosed, for example, in WO 97/45117, U.S. Pat. No. 5,433,955, U.S. Pat. No. 5,614,379 and U.S. Pat. No. 5,624,894. Preferred proteases are aminopeptidases, dipeptidyl aminopeptidases, endoproteases, and endopeptidases. Especially preferred proteases for the release of the active inhibitors from the prodrugs of the present invention are aminopeptidase N, aminopeptidase P, pyroglutaminyl aminopeptidase, dipeptidyl peptidase IV and dipeptidyl peptidase IV-like enzymes.

The released active inhibitors can interact with the DP IV and DP IV-like enzymes. As a direct result, for example, the above-mentioned insulinotropic peptides are broken down to a lesser degree and the effectiveness of insulin is thereby increased.

The administration of unstable inhibitors of DP IV per se has disadvantages since they are degraded very rapidly in vivo and thus an even distribution of the inhibitors, especially in the human body, is impossible. In particular, upon oral administration such inhibitors are so unstable that they have virtually no activity at all. Accordingly, stable inhibitors have hitherto been used especially in the treatment of diabetes mellitus.

In one embodiment, the present invention uses the concept to stabilize unstable inhibitors by masking them in prodrug form.

The properties of the active inhibitors according to the invention can be designed in such a way that the deactivation time of the DP IV-inhibitors e.g. by intramolecular cyclisation after their release from the prodrugs, is definable.

In particular, the prodrugs of the compounds of the invention are advantageous in that the active inhibitors of DP IV and DP IV-like enzymes are released according to individual patients' needs.

When a prodrug of a compound of the invention interacts with a DP IV molecule or a aminopeptidase N molecule, it is cleaved by these enzymes and the active inhibitor is released. The active inhibitor will inhibit DP IV and/or DP IV-like enzymes so that DP IV itself cannot cleave any further compounds for a defined time. The remaining prodrugs are not degraded during this defined time and thus, constitute an inhibitor reservoir until the concentration of DP IV molecules or aminopeptidase N molecules rises again or active inhibitor molecules are eliminated or inactivated.

The use of prodrugs has the further advantage that each organism will release exactly that amount of active inhibitor that is necessary to inhibit that amount of DP IV molecules, which is present in the body of the respective organism.

The present invention accordingly relates to novel compounds of inhibitors of the serine protease dipeptidyl peptidase IV or DP IV-like enzymes and their prodrugs, which can be used in the treatment of various disorders, especially of metabolic disorders associated with diabetes mellitus.

Surprisingly such masked inhibitors are additionally considerably more effective than non-masked inhibitors: if identical amounts of non-masked DP IV-inhibitors and of compounds according to the invention are used, the compounds according to the invention produce a marked improvement in glucose tolerance in Diabetic Zucker rats.

The compounds according to the present invention, are transported through the mucosa of the small intenstine without delay, for example simultaneously with nutrient intake.

Moreover, the site of action, at which the active DP IV-inhibitors are released can also be controlled by the structure of the prodrugs.

To summarise, it may be stated that, using the compounds of the present invention in prodrug form, it is possible in a completely surprising manner:

to achieve increased action of the inhibitors;

to release the active inhibitors according to the patient's needs;

to release the active inhibitors in a temporally controlled manner;

to release the active inhibitors in a site-specific manner; and

to provide a reservoir of DP IV-inhibitors.

As indicated above, the compounds of the present invention, and their corresponding pharmaceutically acceptable acid addition salt forms, are useful in lowering or inhibiting DP IV and DP IV-like enzyme activity at least by about 10, preferably about 50, more preferably about 75, 90 or 100% and prolong the half live of their substrates in a mammal by at least about 2fold, preferably about 3fold, more preferably about 4fold, 5fold or higher relative to the absence of the compound and hence the reduction in the concentration of those peptide hormones and their analogues is reduced or delayed. The ability of the compounds of the present invention, and their corresponding pharmaceutically acceptable acid addition salt forms to inhibit DP IV and DP IV-like enzyme activity may be demonstrated employing the DP IV activity assay for determination of the K₁values and the IC₅₀-values in vitro, as described in examples 8 and 9.

The ability of the compounds of the present invention, and their corresponding pharmaceutically acceptable acid addition salt forms to lower or inhibit DP IV activity in vivo may be demonstrated by oral or intravasal administration to Wistar rats, as described in example 12. The compounds of the present invention inhibit DP IV activity in vivo after both, oral and intravasal administration to Wistar rats.

DP IV is present in a wide variety of mammalian organs and tissues e.g. the intestinal brush-border (Gutschmidt S. et al., “In situ”—measurements of protein contents in the brush border region along rat jejunal villi and their correlations with four enzyme activities. Histochemistry 1981, 72 (3), 467-79), exocrine epithelia, hepatocytes, renal tubuli, endothelia, myofibroblasts (Feller A. C. et al., A monoclonal antibody detecting dipeptidylpeptidase IV in human tissue. Virchows Arch. A. Pathol. Anat. Histopathol. 1986; 409 (2):263-73), nerve cells, lateral membranes of certain surface epithelia, e.g. Fallopian tube, uterus and vesicular gland, in the luminal cytoplasm of e.g., vesicular gland epithelium, and in mucous cells of Brunner's gland (Hartel S. et al., Dipeptidyl peptidase (DPP) IV in rat organs. Comparison of immunohistochemistry and activity histochemistry. Histochemistry 1988; 89 (2): 151-61), reproductive organs, e.g. cauda epididymis and ampulla, seminal vesicles and their secretions (Agrawal & Vanha-Perttula, Dipeptidyl peptidases in bovine reproductive organs and secretions. Int. J. Androl. 1986, 9 (6): 435-52). In human serum, two molecular forms of dipeptidyl peptidase are present (Krepela E. et al., Demonstration of two molecular forms of dipeptidyl peptidase IV in normal human serum. Physiol. Bohemoslov. 1983, 32 (6): 486-96). The serum high molecular weight form of DP IV is expressed on the surface of activated T cells (Duke-Cohan J. S. et al., Serum high molecular weight dipeptidyl peptidase IV (CD26) is similar to a novel antigen DPPT-L released from activated T cells. J. Immunol. 1996, 156 (5): 1714-21).

The compounds of the present invention, and their corresponding pharmaceutically acceptable acid addition salt forms are able to inhibit DP IV in vivo. In one embodiment of the present invention, all molecular forms, homologues and epitopes of DP IV from all mammalian tissues and organs, also of those, which are undiscovered yet, are intended to be embraced by the scope of this invention.

In another preferred embodiment of the present invention, all molecular forms, homologues and epitopes of proteins comprising DP IV-like enzyme activity, from all mammalian tissues and organs, also of those, which are undiscovered yet, are intended to be embraced by the scope of this invention.

The ability of the compounds of the present invention, and their corresponding pharmaceutically acceptable acid addition salt forms to lower or inhibit the activity of DP IV-like enzymes may be demonstrated employing an enzyme activity assay for determination of the K₁-values in vitro as described in example 10.

In another embodiment, the compounds of the present invention, and their corresponding pharmaceutically acceptable acid addition salt forms have only low, if no inhibitory activity against non-DP IV and non-DP IV-like proline specific enzymes. See example 11.

In view of their ability to inhibit DP IV and DP IV-like enzyme activity, the compounds of the present invention, and their corresponding pharmaceutically acceptable acid addition salt forms, are useful in treating conditions mediated by said enzyme activities. Based on the findings described in the examples of the present invention and in the literature, it can be shown that the compounds disclosed herein are useful in the treatment of conditions such as immune, autoimmune disorders or central nervous system disorders, selected from the group consisting of strokes, tumors, ischemia, Parkinson's disease, and migraines.

In a more preferred embodiment of this invention, the compounds of the present invention and their corresponding pharmaceutically acceptable acid addition salt forms, improve glucose tolerance by lowering elevated blood glucose levels in response to an oral glucose challenge and, therefore, are useful in treating non-insulin-dependent diabetes mellitus. The ability of the compounds of the present invention, and their corresponding pharmaceutically acceptable acid addition salt forms, to improve glucose tolerance in response to an oral glucose challenge, may be measured in diabetic Zucker rats. The method is described in example 13.

The present invention therefore provides a method of preventing or treating a condition mediated by modulation of the DP IV or DP IV-like enzyme activity in a subject in need thereof which comprises administering any of the compounds of the present invention or pharmaceutical compositions thereof in a quantity and dosing regimen therapeutically effective to treat the condition. Additionally, the present invention includes the use of the compounds of this invention, and their corresponding pharmaceutically acceptable acid addition salt forms, for the preparation of a medicament for the prevention or treatment of a condition mediated by modulation of the DP IV activity in a subject. The compound may be administered to a patient by any conventional route of administration, including, but not limited to, intravenous, oral, subcutaneous, intramuscular, intradermal, parenteral and combinations thereof.

In a further preferred form of implementation, the invention relates to pharmaceutical compositions, that is to say, medicaments, that contain at least one compound of the invention or salts thereof, optionally in combination with one or more pharmaceutically acceptable carriers and/or solvents.

The pharmaceutical compositions may, for example, be in the form of parenteral or enteral formulations and contain appropriate carriers, or they may be in the form of oral formulations that may contain appropriate carriers suitable for oral administration. Preferably, they are in the form of oral formulations.

The pharmaceutical compositions may additionally contain one or more hypoglycaemically active ingredients which may be active ingredients that are known per se.

The inhibitors or prodrugs of DP IV and DP IV-like enzymes administered according to the invention may be employed in pharmaceutically administrable formulations or formulation complexes alone or in combination with DP IV-inhibitors, substrates or pseudosubstrates of DP IV or DP IV-like enzymes, inhibitors of DP IV or DP IV-like enzyme expression, binding proteins of or antibodies against DP IV and DP IV-like enzymes in mammals. The compounds of the invention make it possible to adjust treatment individually to patients and diseases, it being possible, in particular, to avoid individual intolerances, allergies and side-effects.

The compounds also exhibit differing degrees of activity as a function of time. Thus it is thereby possible to respond differently to the individual situation of patients: on the one hand it is possible to precisely adjust the speed of the onset of action and, on the other hand, the duration of action and especially the intensity of action.

The method according to the invention represents especially a new approach to the reduction of raised blood glucose concentration in the serum of mammals. It is simple, susceptible of commercial application and suitable for use in the treatment of especially diseases that are based on above-average blood glucose values, on neurodegenerative diseases or on high blood pressure, in mammals and especially in human medicine.

The compounds are administered, for example, in the form of pharmaceutical preparations that contain the active ingredient in combination with customary additives like diluents, excipients and/or carriers known from the prior art. For example, they are administered parenterally (for example i.v. in physiological saline solution) or enterally (for example orally, formulated with customary carriers, such as, for example, glucose).

Depending upon their endogenous stability and their bioavailability, one or more doses of the compounds can be given per day in order to achieve the desired normalisation of the blood glucose values. For example, such a dosage range in humans may be in the range of from 0.01 mg to 250.0 mg of compound per kilogram body weight per day, preferably in the range of from 0.01 to 100 mg of compound per kilogram of body weight per day.

It has been found that by administering inhibitors of dipeptidyl peptidase IV and DP IV-like enzyme activities in the blood of a mammal, owing to the associated temporary reduction in activity, the endogenous (or additionally exogenously administered) insulinotropic peptides Gastric Inhibitory Polypeptide 1-42 (GIP-₁₋₄₂) and Glucagon-Like Peptide Amide-1 7-36 (GLP-1₇₋₃₆) (or other GLP-1₇₋₃₇ or analogues thereof) are, as a consequence, broken down to a lesser extent by DP IV and DP IV-like enzymes and hence the reduction in the concentration of those peptide hormones and their analogues is reduced or delayed. The increased stability of the (endogenous or exogenously supplied) incretins or their analogues, which is achieved owing to the action of DP IV inhibitors and which results in their being available in greater quantities for insulinotropic stimulation of the incretin receptors of the Langerhans cells in the pancreas, alters inter alia the effectiveness of the body's own insulin, which brings with it a stimulation of the carbohydrate metabolism of the subject treated.

As a result, the blood sugar level decreases by about 10%, preferably by about 15%, more preferably by about 20 or 30% in the serum of the hyperglycaemic subject treated, compared to the untreated subject. Most preferably, the blood sugar level of a subject is reduced down to a level below 140, especially preferred between 60 and 100 mg glucose/dl in the postprandial phase or below 100, preferably down to a level between 60 and 80 mg glucose/dl in the fasting state.

Accordingly, it is possible to prevent or alleviate metabolic abnormalities, such as impaired glucose tolerance, glucosuria, hyperlipidaemia, metabolic acidosis, diabetes mellitus, diabetic neuropathy and nephropathy and sequelae caused by diabetes mellitus in mammals, metabolism-related hypertension and cardiovascular sequelae caused by hypertension in mammals, skin diseases and diseases of the mucosae, autoimmune diseases, high blood pressure and inflammatory conditions, and psychosomatic, neuropsychiatric and depressive illnesses, such as anxiety, depression, sleep disorders, chronic fatigue, schizophrenia, epilepsy, nutritional disorders, spasm and chronic pain.

To enhance the blood-sugar-reducing action of various antidiabetics, combinations of various orally active antidiabetics are often used. Since the antihyperglycaemic action of the compounds of the invention operates independently of other known orally administrable antidiabetics, the active ingredients of the invention are analogously suitable for use in combination therapies, in an appropriate galenical form, for achieving the desired normoglycaemic effect.

The compounds used according to the invention can accordingly be converted in a manner known per se into conventional formulations, such as, for example, tablets, capsules, dragées, pills, suppositories, granules, aerosols, syrups, liquid, solid and cream-like emulsions and suspensions and solutions, using inert, non-toxic, pharmaceutically suitable carriers and additives or solvents. In each of those formulations, the therapeutically effective compounds are preferably present in a concentration of approximately from 0.1 to 80% by weight, preferably from 1 to 50% by weight, of the total mixture, that is to say, in amounts sufficient for the mentioned dosage latitude to be obtained.

The good absorption of the compounds used according to the invention by the mucosae of the gastrointestinal tract makes it possible for many galenical preparations to be used:

The substances can be used as medicaments in the form of dragées, capsules, bitable capsules, tablets, drops, syrups or also as suppositories or as nasal sprays.

The formulations are prepared, for example, by extending the active ingredient with solvents and/or carriers, optionally with the use of emulsifiers and/or dispersants, it being possible, for example, in the case where water is used as diluent, for organic solvents to be optionally used as auxiliary solvents.

There may be mentioned as examples of excipients: water, non-toxic organic solvents, such as paraffins (for example natural oil fractions), vegetable oils (for example rapeseed oil, groundnut oil, sesame oil), alcohols (for example ethyl alcohol, glycerol), glycols (for example propylene glycol, polyethylene glycol); solid carriers, such as, for example, natural powdered minerals (for example highly disperse silica, silicates), sugars (for example raw sugar, lactose and dextrose); emulsifiers, such as non-ionic and anionic emulsifiers (for example polyoxyethylene fatty acid esters, polyoxyethylene fatty alcohol ethers, alkylsulphonates and arylsulphonates), dispersants (for example lignin, sulphite liquors, methylcellulose, starch and polyvinylpyrrolidone) and lubricants (for example magnesium stearate, talcum, stearic acid and sodium lauryl sulphate) and optionally flavourings.

Administration is carried out in the usual manner, preferably enterally or parenterally, especially orally. In the case of enteral administration, tablets may contain in addition to the mentioned carriers further additives such as sodium citrate, calcium carbonate and calcium phosphate, together with various additives, such as starch, preferably potato starch, gelatin and the like. Furthermore, lubricants, such as magnesium stearate, sodium lauryl sulphate and talcum, can be used concomitantly for tabletting. In the case of aqueous suspensions and/or elixirs intended for oral administration, various taste correctives or colourings can be added to the active ingredients in addition to the above-mentioned excipients.

In the case of parenteral administration, solutions of the active ingredients using suitable liquid carriers can be employed. In general, it has been found advantageous to administer, in the case of intravenous administration, amounts of approximately from 0.01 to 2.0 mg/kg, preferably approximately from 0.01 to 1.0 mg/kg, of body weight per day to obtain effective results and, in the case of enteral administration, the dosage is approximately from 0.01 to 2 mg/kg, preferably approximately from 0.01 to 1 mg/kg, of body weight per day.

It may nevertheless be necessary in some cases to deviate from the stated amounts, depending upon the body weight of the experimental animal or the patient or upon the type of administration route, but also on the basis of the species of animal and its individual response to the medicament or the interval at which administration is carried out. Accordingly, it may be sufficient in some cases to use less than the above-mentioned minimum amount, while, in other cases, the mentioned upper limit will have to be exceeded. In cases where relatively large amounts are being administered, it may be advisable to divide those amounts into several single doses over the day. For administration in human medicine, the same dosage latitude is provided. The above remarks apply analogously in that case.

### EXAMPLES OF PHARMACEUTICAL FORMULATIONS

1. Capsules containing 100 mg of a compound of the invention per capsule:

For approximately 10,000 capsules a solution of the following composition is prepared:

| | |
|---|---|
| compound of the invention | 1.0 kg |
| glycerol | 0.5 kg |
| polyethylene glycol | 3.0 kg |
| water | 0.5 kg |
| | 5.0 kg |

The solution is introduced into soft gelatin capsules in a manner knownper se. The capsules are suitable for chewing or swallowing.

2. Tablets or coated tables or dragees containing 100 mg of a compound of the invention:

The following amounts refer to the preparation of 100,000 tablets:

| | |
|---|---|
| compound of the invention, | 10.0 kg |
| finely ground | |
| glucose | 4.35 kg |
| lactose | 4.35 kg |
| starch | 4.50 kg |
| cellulose, finely ground | 4.50 kg |

The above constituents are mixed and then provided with a solution prepared from

| | |
|---|---|
| polyvinylpyrrolidone | 2.0 kg |
| polysorbate | 0.1 kg |
| and water approx. | 5.0 kg |

and granulated in a manner known per se by grating the moist mass and, after the addition of 0.2 kg of magnesium stearate, drying it. The finished tablet mixture of 30.0 kg is processed to form convex tablets weighing 300 mg. The tablets can be coated or sugar-coated in a manner known per se.

### EXAMPLES OF THE INVENTION

### Example 1

### Synthesis of substituted aminoketones

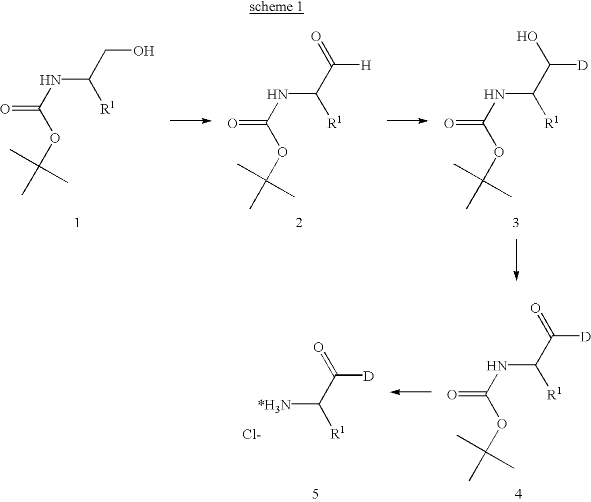

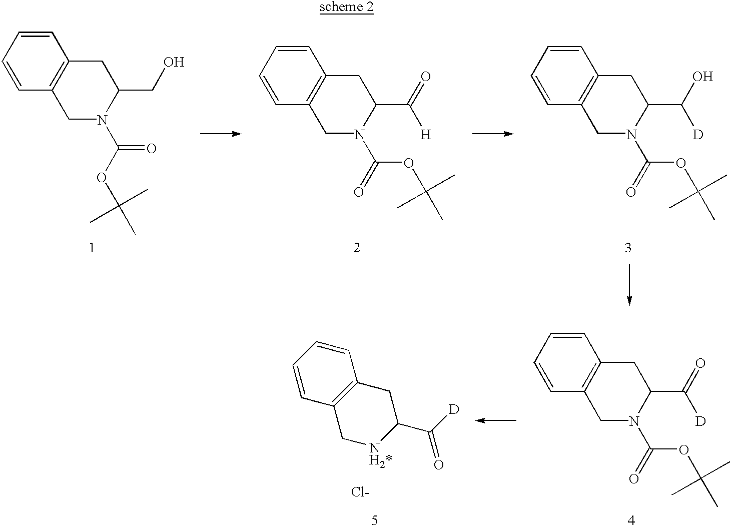

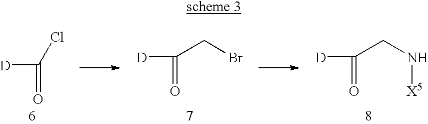

### Example 1 (scheme 1)

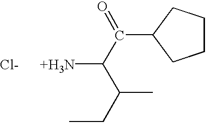

Boc-isoleucinal 2

Oxalylchloride (714 μl, 8.28 mmol) was dissolved 10 ml of dry dichlormethane and brought to −78° C. Then DMSO (817 μl, 8.28 mmol) was added dropwise. The solution was stirred for 20 min at −78° C. Then 1 (1.00 g, 4.6 mmol) was added and the mixture was stirred for 20 min. After that TEA (2.58 ml, 18.4 mmol) was added and the mixture was allowed to reach r.t. The mixture was diluted with hexane/ethylacetate (2/1 v/v) and 10 ml of HCl (10% in water) was added. The organic layer was separated and the aqueous phase was extracted with 20 ml of methylenechloride. All organic layers where collected and whashed with brine, followed by water, then dried.

The product was purified by column chromatography using silica gel and heptane/chloroformn.

Yield: 0.52 g, 52%

tert-butyl N-1-[cyclopentyl(hydroxy)methyl]-2-methylbutylcarbamate 3

2 (0.52 g, 2.42 mmol) was dissolved in 10 ml of dry THF and cooled down to 0° C. Then cyclopentylmagnesiumbromide (1.45 ml of a 2M solution) where added. After completion of the reaction water (2 ml) was added and solution was neutralized by adding aqueous HCl. Then methylenechloride was added and the organic layer was separated and dried (Na₂SO₄). After evaporation the resulting oil was used without further characterization.

tert-butyl N-[1-(cyclopentylcarbonyl)-2-methylbutyl]carbamate 4

3 (0.61 g, 2.15 mmol) was treated like l. Oxalylchloride (333 μl, 3.87 mmol), DMSO (382 μl, 5.37 mmol), TEA (1.2 ml, 8.59 mmol)

Yield: 0.180g, 30%

1-cyclopentyl-3-methyl-1-oxo-2-pentanaminium chloride 5

4 (0.18 g, 0.63 mmol) was dissolved in 2 ml HCl (7N in dioxane). After completion of the reaction the solvent was removed and the resulting oil was purified by column chromatography on silical gel using a chloroform/methanol/water gradient. The resulting oil was triturated with ether. Yield: 0.060 g, ¹H-NMR: (500 MHz, CDCl₃), 01=0.85-0.90 (m, 1H), 0.91-0.95 (t, 3H), 0.98-1.15 (m, 1H), 1.09-1.12 (d, 3H), 1.22-1-31 (m, 2H), 1.81-1.90 (m, 1H), 1.91-1.99 (m, 1H), 2.09-2.189 (m, 1H), 2.95-3.05 (m, 1H), 4.17-4.19 (d, 1H), 8.41-8.61 (br. s 3H), ESI-MS: m/z=184.2 (M+H)

### Example 2 (scheme 1)

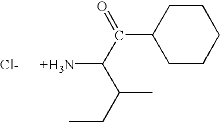

For the synthesis procedure refer to example 1, using cyclohexylmagnesuimbromidbromide for step 3

Yield: 0.100 g, ¹H-NMR: (500 MHz, CDCl₃), □=0.91-0.95 (t, 3H), 1.15-1.2 (d, 3H), 1.21-1.29 (m, 3H), 1.33-1.39 (m, 2H), 1.45-1.55 (m, 1H), 1.61-1.69 (m, 2H), 1.72-1.81 (m, 2H), 1.95-2.05 (m, 1H), 2.09-2.18 (m, 1H), 2.45-2.55 (m, 1H), 4.25-4.31 (m, 1H), 8.41-8.61 (br. s 3H), ESI-MS:m/z=198.3 (M+H)

### Example 3 (scheme 1)

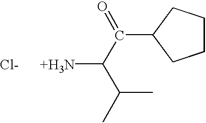

For the synthesis procedure refer to example 1, using valinol for step 1.

Yield: 0.130 g, ¹H-NMR: (500 MHz, CDCl₃), □=0.71-0.80 (m, 4H), 1.31-1.42 (m, 1H), 1.65-1.70 (d, 6H), 2.19-1.25 (m, 4H), 2.81-2.91 (m, 1H), 4.15-4.20 (m, 1H), 8.41-8.61 (br. s 3H), ESI-MS:m/z=170.3 (M+H)

### Example 4 (scheme 1)

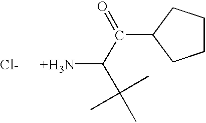

For the synthesis procedure refer to example 1, using tert-butyl-Ile for step 1.

Yield: 0.05 g, ¹H-NMR: (500 MHz, CDCl₃), □=0.89-0.97 (m, 4H), 1.59-1.61 (s, 9H), 2.21-2.29 (m, 4H), 2.95-3.01 (m, 1H), 4.45-4.49 (m, 1H), 8.41-8.61 (br. s 3H), ESI-MS:m/z=184.3 (M+H)

### Example 5 (scheme 1)

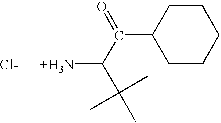

For the synthesis procedure refer to example 1, using N-Boc-tert-butyl-Isoleucinole for step 1 and cyclohexylmagnesiumbromide for step 3.

Yield: 0.06 g, ¹H-NMR: (500 MHz, CDCl₃), □=0.99-1.25 (m, 13H), 1.59-1.82 (m, 5H), 2.45-2.55 (m, 1H), 4.01-4.09 (m, 1H), 8.51-8.61 (br. s 3H), ESI-MS:m/z=198.3 (M+H)

### Example 6 (scheme 2)

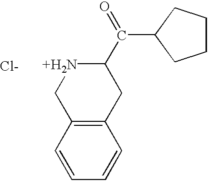

For the synthesis procedure refer to example 1, using N-Boc-2-hydroxymethyltetraisoquinoline for step 1.

Yield: 0.95 g, ¹H-NMR: (500 MHz, CDCl₃), □=1.21-1.99 (m, 8H), 3.01-3.15 (m, 1H), 3.25-3.42 (m, 2H), 4.31-4.45 (m, 2H), 4.61-4.71 (m, 1H) 7.06-7.21 (m, 4H), 9.75-9.85 (br.s., 1H), 10.75-10.85 (bs., 1H), ESI-MS:m/z=230.2 (M+H)

### Example 7 (scheme 3)

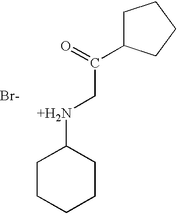

Brommethyl-cyclohexylketone 6

Cyclopentancarboxylic acid chloride 5 (1.00 g, 7.54 mmol) was dissolved in 5 ml of dry ethyl ether and the solution was brought to −20° C. Then diazomethane (37.7 mmol in 50 ml dry ether) was added dropwise. The mixture was allowed to stirr at −30° C. for 1.5 h followed by 1.5 h at 0° C. After that HBr (33% in acetic acid) (2.01 ml, 11.3 mmol) was added and the solution stirred for 30 min at r.t. The solution was diluted by adding 50 ml of ether and extracted using brine. The organic layer was dried and evaporated and the product was used without further characterisation.

N-(2-cyclopentyl-2-oxoethyl)cyclohexanaminium bromide 7

6 (1.27 g, 6.67 mmol) was dissolved in 12 ml of acetonitrile/chloroforme (1/1, v/v) and cooled down to 0° C. Then cyclohexylamine (762 μl, 6.67 mmol) was added dropwise. The suspension formed was stirred for 1 h at r.t. Then the white precipitate formed was filtered off. The filtrate was concentrated and ether was added. The resulting white solid was filtered and dried.

Yield: 0.3 g, ESI-MS:m/z=210.2 (M+H)

Especially synthesized compounds of the invention are:

example 2: 1-cyclopentyl-3-methyl-1-oxo-2-pentanaminium chloride

example 3: 1-cyclopentyl-3-methyl-1-oxo-2-butanaminium chloride

example 4: 1-cyclopentyl-3,3-dimethyl-1-oxo-2-butanaminium chloride

example 5: 1-cyclohexyl-3,3-dimethyl-1-oxo-2-butanaminium chloride

example 6: 3-(cyclopentylcarbonyl)-1,2,3,4-tetrahydroisoquinolinium chloride

example 7: N-(2-cyclopentyl-2-oxoethyl)cyclohexanaminium chloride

From the compounds of the present invention biological efficacy data were investigated. The methods are described in the further examples.

### Example 8: Kᵢ-determination

In order to measure the inhibition constant Kᵢ, a photometric assay was used The test compounds were measured as competitors of the standard substrate GP-4-Nitroanilide. Three different substrate concentrations (0.4 mM to 0.05mM) were combined with 8 different competitor concentrations (0.5 mM to 2 μM). The reaction was started by addition of 3.5 nM DP IV. Experiments were carried out under standard conditions: 30° C. in pH 7.6 40 mM HEPES (Sigma-Aldrich) buffer. Nitroaniline production was monitored using a HTS 7000+microplate reader (PerkinElmer, Überlingen, Germany). The K₁-values were calculated via non-linear regression using the enzyme kinetic program Grafit 4.016 (Erithacus Ltd, UK).

For a reversible competitive inhibition is to assumed:

$${v}_{i} = \frac{{V}_{max} * {K}_{m}}{[ S ] + {K}_{m} ( 1 + \frac{[ I ]}{{K}_{1}} )}$$

$$Legend . [ I ] inhibitor concentration$$

$${K}_{1} inhibition constant$$

For the compound 1-cyclopentyl-3-methyl-1-oxo-2-pentanaminium chloride a Kᵢ-value of 6.29*10⁶ was determined.

### Example 9

### Determination of IC₅₀-Values

100 μl inhibitor stock solution were mixed with 100 μl buffer (HEPES pH7.6) and 20 μl diluted porcine DP IV and preincubated at 30° C. Reaction was started by addition of a mixture of 50 μl substrate (Gly-Pro-pNA, final concentration 0.4 mM) and 2 μl APN stock solution. Formation of the product pNA was measured at 405 nm and 30° C. over 10 min using the HTS 7000Plus plate reader (Perkin Elmer) and slopes were calculated. The final inhibitor concentrations ranged between 1 mM and 30 nM. For calculation of IC50 GraFit 4.0.13 (Erithacus Software) was used.

### Example 10

### Inhibition Of DPIV-Like Enzymes—Dipeptidyl Peptidase II (DP II)

DP II (3.4.14.2) releases N-terminal dipeptides from oligopeptides if the N-terminus is not protonated (McDonald, J. K., Ellis, S. & Reilly, T. J., 1966, J. Biol. Chem., 241, 1494-1501). Pro and Ala in P₁-position are preferred residues. The enzyme activity is described as DP IV-like activity, but DP II has an acidic pH-optimum. The enzyme used was purified from porcine kidney.

Assay:

100 μl inhibitor in an concentration range of 1*10⁻⁴M−5*10⁻⁸M were admixed with 100 μl μl buffer solution (40 mM HEPES, pH7.6, 0.015% Brij, 1 mM DTT), 50 μl lysylalanylaminomethylcoumarine solution (5 mM) and 20 μl porcine DP II (250 fold diluted in buffer solution). Fluorescence measurement was performed at 30° C. and λ_{excitation}=380 nm, λₑₘᵢₛₛᵢₒₙ=465 nm for 25 min using a plate reader (HTS7000plus, Applied Biosystems, Weiterstadt, Germany). The Kᵢ-values were calculated using Graphit 4.0.15 (Erithacus Software, Ltd., UK).

Attractin

100 μl inhibitor stock solution were mixed with 100 μl buffer (HEPES pH7.6) and 20 μl diluted attractin and preincubated at 30° C. Reaction was started by addition of a mixture of 50 μl substrate (Gly-Pro-pNA, final concentration 0.4 mM) and 2 μl APN stock solution. Formation of the product pNA was measured at 405 nm and 30° C. over 10 min using the HTS 7000Plus plate reader (Perkin Elmer) and slopes were calculated. The final inhibitor concentrations ranged between 1 mM and 30 nM. For calculation of IC₅₀ values, GraFit 4.0.13 (Erithacus Software) was used.

### Example 11

### Cross Reacting Enzymes

The inhibitors were tested for their cross reacting potency against dipeptidyl peptidase I, prolyl oligopeptidase and Prolidase.

Dipeptidyl peptidase I (DP I, cathepsin C):

DP I or cathepsin C is a lysosomal cysteine protease which cleaves off dipeptides from the N-terminus of their substrates (Gutman, H. R. & Fruton, J. S., 1948, J. Biol: Chem., 174, 851-858). It is classified as a cysteine protease. The enzyme used was purchased from Qiagen (Qiagen GmbH, Hilden, Germany). In order to get a fully active enzyme, the enzyme was diluted 1000fold in MES buffer pH5,6 (40 mM MES, 4 mM DTT, 4 mM KCl, 2 mM EDTA, 0.015% Brij) and pre-incubated for 30 min at 30° C.

Assay:

50 μl solution with the test compounds in a concentration range of 1*10⁻⁵M−1*10⁻⁷M were admixed with 110 μl buffer-enzyme-mixture. The assay mixture was pre-incubated at 30° C. for 15 min. After pre-incubation, 100 μl histidylseryl-βnitroaniline (2*10⁻⁵M) was added and measurement of yellow color development due to β-nitroaniline release was performed at 30° C. and λ_{excitation}=380 nm, λₑₘᵢₛₛᵢₒₙ=465 nm for 10 min., using a plate reader (HTS7000plus, Applied Biosystems, Weiterstadt, Germany).

The IC₅₀-values were calculated using Graphit 4.0.15 (Erithacus Software, Ltd., UK).

Prolidase (X-Pro dipeptidase)

Prolidase (EC 3.4.13.9) was first described by Bergmann & Fruton (Bergmann, M. & Fruton, J S, 1937, J. Biol. Chem. 189-202). Prolidase releases the N-terminal amino acid from Xaa-Pro dipeptides and has a pH optimum between 6 and 9.

Prolidase from porcine kidney (ICN Biomedicals, Eschwege, Germnany). was solved (1 mg/ml) in assay buffer (20 mM NH₄(CH₃COO)₂, 3 mM MnCl₂, pH 7.6). In order to get a fully active enzyme the solution was incubated for 60 min at room temperature.

Assay:

450 μl solution with the test compounds in an concentration range of 5*10⁻³M−5*10⁻⁷M were admixed with 500 μl buffer solution (20 mM NH₄(CH₃COO)₂, pH 7.6) and 250 μl Ile-Pro-OH (0.5 mM in the assay mixture). The assay mixture was pre-incubated at 30° C. for 5 min. After pre-incubation, 75 μl Prolidase (1:10 diluted in assay buffer) were added and measurement was performed at 30° C. and λ=220 nm for 20 min using a UV/Vis photometer, UV1 (Thermo Spectronic, Cambridge, UK). The IC₅₀-values were calculated using Graphit 4.0.15 (Erithacus Software, Ltd., UK).

Angiotensin-I converting enzyme (ACE)

Angiotensin I-converting enzyme (ACE; peptidyl-dipeptidase A) is a zinc metallopeptidase which cleaves the C-terminal dipeptide from angiotensin I to produce the potent vasopressor octapeptide angiotensin II (Skeggs L. T., Kahn, J. R. & Shumway, N. P. (1956) The preparation and function of the hypertensin-converting enzyme. J. Exp. Med. 103, 295-299.) and inactivates bradykinin by the sequential removal of two C-terminal dipeptides (YangH. Y. T., Erdös, E. G. & Levin, Y. (1970) A dipeptidyl carboxypeptidase that converts angiotensin I and inactivates bradykinin. Biochim. Biophys. Acta 214, 374-376.). In addition to these two main physiological substrates, which are involved in blood pressure regulation and water and salt metabolism, ACE cleaves C-terminal dipeptides from various oligopeptides with a free C-terminus. ACE is also able to cleave a C-terminal dipeptide amide.

Assay:

For IC₅₀ determination of ACE an enzyme produced by Sigma was used (Prod.No. A-6778). The assay procedure and calculation of activity described by the manufacturer was adapted to half of the described volumes.

The IC₅₀-values were calculated using Graphit 4.0.15 (Erithacus Software, Ltd., UK).

Acylamino acid-releasing enzyme (AARE)

Acylaminoacyl-peptidase (EC 3.4.19.1) has also been referred to by the names acylpeptide hydrolase (Gade W. & Brown, J. L. (1978) Purification and partial characterization of a-N-acylpeptide hydrolase from bovine liver. J. Biol. Chem. 253, 5012-5018; Jones W. M. & Manning, J. M. (1985) Acylpeptide hydrolase activity from erythrocytes. Biochem. Biophys. Res. Commun. 126, 933-940.; KobayashiK., Lin, L. -W., Yeadon, J. E., Klickstein, L. B. & Smith, J. A. (1989) Cloning and sequence analysis of a rat liver cDNA encoding acylpeptide hydrolase. J. Biol. Chem. 264, 8892-8899), acylamino acid-releasing enzyme (Tsunasawa S., Narita, K. & Ogata, K. (1975) Purification and properties of acylamino acid-releasing enzyme from rat liver. J. Biochem. 77, 89-102.; Mitta M., Asada, K., Uchimura, Y., Kimizuka, F., Kato, I., Sakiyama, F. & Tsunasawa, S. (1989) The primary structure of porcine liver acylamino acid-releasing enzyme deduced from cDNA sequences. J. Biochem. 106, 548-551.) and acylaminoacyl peptide hydrolase (Radhakrishna G. & Wold, F. (1989) Purification and characterization of an N-acylaminoacyl-peptide hydrolase from rabbit muscle. J. Biol. Chem. 264, 11076-11081.). Acylaminoacyl peptidase catalyzes the removal of an N-acylated amino acid from a blocked peptide: Block-Xaa↓Xbb-Xcc. . . . The products of the reaction are the free acyl amino acid and a peptide with a free N-terminus shortened by one amino acid. The enzyme acts on a variety of peptides with different N-terminal acyl groups, including acetyl, chloroacetyl, formyl and carbamyl (Jones W. M., Scaloni, A., Bossa, F., Popowicz, A. M., Schneewind, O. & Manning, J. M. (1991) Genetic relationship between acylpeptide hydrolase and acylase, two hydrolytic enzymes with similar binding but different catalytic specificities. Proc. Natl Acad. Sci. USA 88, 2194-2198.).

Assay:

100 μl solution with the inhibitors in an concentration range of 1*10⁻⁴M−5*10⁻⁸M were admixed with 100 μl μl buffer solution (200 mM Natriumphosphat, pH 7.2) and 20 μl AARE solution. The assay mixture was pre-incubated at 30° C. for 15 min. After pre-incubation, 50 μl Acetyl-Met-AMC solution (0.54 mM) was added. Release of the AMC was measured at 30° C. using a Novovostar flourescence microplate reader (BMG) and excitation/emission wavelengths of 380/460 nm.

The IC₅₀-values were calculated from the slopes of the progress curves using Graphit 4.0.15 (Erithacus Software, Ltd., UK).

### Example 12

### Determination Of DP IV Inhibiting Activity After Intravasal And Oral Administration To Wistar Rats

Animals

Male Wistar rats (Shoe: Wist(Sho)) with a body weight ranging between 250 and 350 g were purchased from Tierzucht Schönwalde (Schönwalde, Germany).

Housing conditions

Animals were single-caged under conventional conditions with controlled temperature (22±2° C.) on a 12/12 hours light/dark cycle (light on at 06:00 AM). Standard pelleted chow (ssniff® Soest, Germany) and tap water acidified with HCl were allowed ad libitum.

Catheter insertion into carotid artery

After ≧one week of adaptation at the housing conditions, catheters were implanted into the carotid artery of Wistar rats under general anaesthesia (i.p. injection of 0.25 ml/kg b.w. Rompun® [2%], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week. In case of catheter dysfunction, a second catheter was inserted into the contra-lateral carotid artery of the respective rat. After one week of recovery from surgery, this animal was reintegrated into the study. In case of dysfunction of the second catheter, the animal was withdrawn from the study. A new animal was recruited and the experiments were continued in the planned sequence, beginning at least 7 days after catheter implantation.

Experimental design

Rats with intact catheter function were administered placebo (1 ml saline, 0.154 mol/l) or test compound via the oral and the intra-vasal (intra-arterial) route.

After overnight fasting, 100 μl samples of heparinised arterial blood were collected at −30, −5, and 0 min. The test substance was dissolved freshly in 1.0 ml saline (0.154 mol/l) and was administered at 0 min either orally via a feeding tube (75 mm; Fine Science Tools, Heidelberg, Germany) or via the intra-vasal route. In the case of oral administration, an additional volume of 1 ml saline was injected into the arterial catheter. In the case of intra-arterial administration, the catheter was immediately flushed with 30 μl saline and an additional 1 ml of saline was given orally via the feeding tube.

After application of placebo or the test substances, arterial blood samples were taken at 2.5, 5, 7.5, 10, 15, 20, 40, 60 and 120 min from the carotid catheter of the conscious unrestrained rats. All blood samples were collected into ice cooled Eppendorf tubes (Eppendorf-Netheler-Hinz, Hamburg, Germany) filled with 10 μl 1M sodium citrate buffer (pH 3.0) for plasma DP IV activity measurement. Eppendorf tubes were centrifuged immediately (12000 rpm for 2 min, Hettich Zentrifuge EBA 12, Tuttlingen; Germany): The plasma fractions were stored on ice until analysis or were frozen at −20° C. until analysis. All plasma samples were labelled with the following data:

Code number

Animal Number

Date of sampling

Time of sampling

Analytical Methods

The assay mixture for determination of plasma DP IV activity consisted of 80 μl reagent and 20 μl plasma sample. Kinetic measurement of the formation of the yellow product 4-nitroaniline from the substrate glycylprolyl-4-nitroaniline was performed at 390 nm for 1 min at 30° C. after 2 min pre-incubation at the same temperature. The DP IV activity was expressed in mU/ml.

Statistical methods

Statistical evaluations and graphics were performed with PRISM® 3.02 (GraphPad Software, Inc.). All parameters were analysed in a descriptive manner including mean and SD.

### Example 13

### The effect of substituted amino ketones on glucose tolerance in diabetic Zucker rats

Study Design

Animals

N=30 male Zucker rats (fa/fa), mean age 11 weeks (5-12 weeks), mean body weight 350 g (150-400 g), were purchased from Charles River (Sulzfeld, Germany). They were kept for >12 weeks until all the fatty Zucker rats had the characteristics of manifest Diabetes mellitus.

Housing conditions

Animals were kept single-housed under conventional conditions with controlled temperature (22±2° C.) on a 12/12 hours light/dark cycle (light on at 06:00 a.m.). Standard pellets (ssniff®, Soest, Germany) and tap water acidified with HCl were allowed ad libitum.

Catheterization of carotid artery

Fatty Zucker rats, 17-24 weeks old, adapted to the housing conditions, were well prepared for the tests. Catheters were implanted into the carotid artery of fatty Zucker rats under general anaesthesia (i.p. injection of 0.25 ml/kg b.w. Rompun®[2%], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week.

In case of catheter dysfunction, a second catheter was inserted into the contra-lateral carotid artery of the respective rat. After one week of recovery from surgery, this animal was reintegrated into the study. In case of dysfunction of the second catheter, the animal was withdrawn from the study. A new animal was recruited and the experiments were continued in the planned sequence, beginning at least 7 days after catheter implantation.

Experimental design

Fatty Zucker rats with intact catheter function were given in random order placebo (1 ml saline, 0.154 mol/l; N=9 animals as control), or test substance, solved in 1 ml saline (N=6 animals in each test group).

After overnight fasting, the fatty Zucker rats were given placebo and test substance, respectively, via feeding tube orally (15 G, 75 mm; Fine Science Tools, Heidelberg, Germany) at −10 min. An oral glucose tolerance test (OGTT) with 2 g/kg b.w. glucose as a 40% solution (B. Braun Melsungen, Melsungen, Germany) was implemented at ±0 min. The glucose was administered via a second feeding tube. Arterial blood samples from the carotid catheter were collected at −30 min, −15 min, ±0 min and at 5, 10, 15, 20, 30, 40, 60, 90 and 120 min into 20 μl glass capillaries, which were placed in standard tubes filled with 1 ml solution for hemolysis (blood glucose measurement).

In addition, arterial blood samples were taken at −30 min, at 20, 40 60 and 120 min from the carotid catheter of the conscious unrestrained fatty Zucker rats and given into ice cooled Eppendorf tubes (Eppendorf-Netheler-Hinz, Hamburg, Germany) filled with 10 μl sodium citrate buffer (pH 3.0) for plasma DP activity measurement. Eppendorf tubes were centrifuged immediately (12000 rpm for 2 min, Hettich Zentrifuge EBA 12, Tuttlingen; Germany): The plasma fractions were stored on ice until analysis.

Analytical Methods

Blood glucose: Glucose levels were measured using the glucose oxidase procedure (Super G Glukosemeβgerät; Dr. Müller Gerätebau, Freital, Germany).

The compounds of the present invention, tested in the in vivo assay, improved significantly the glucose tolerance after oral administration during an OGTT in Zucker rats (see 7.1).

1. compounds of the general formula I
B—(CH—R¹)ₙ—C(=X²)—D (I)
wherein
n is 0 or 1,
R¹ stands for H, C₁-C₉ branched or straight chain alkyl, n-butan-2-yl, n-prop-2-yl or isobutyl, C₂-C₉ branched or straight chain alkenyl, C₃-C₈ cycloalkyl, C₅-C₇ cycloalkenyl, aryl, heteroaryl or a side chain of a natural amino acid or derivatives thereof,
X² stands for O,NR⁶, N⁺(R⁷)₂, or S,
B is selected from the following groups: 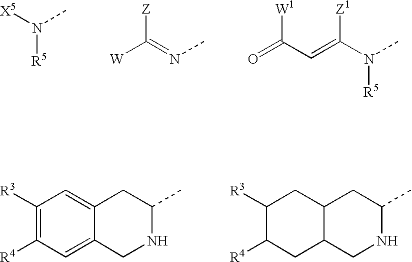
where X⁵ is H or an acyl or oxycarbonyl group including amino acids,
R⁵ is H, C₁-C₉ branched or straight chain alkyl, C₂-C₉ branched or straight chain alkenyl, C₃-C₈ cycloalkyl, C₅-C₇ cycloalkenyl, aryl, heteroaryl or a side chain of a natural amino acid or mimetics thereof, or a group of the formula —(CH)ₘ—NH—C₅H₃N—Y where m is an integer of 2-4, —C₅H₃N—Y is a divalent pyridyl moiety and Y is a hydrogen atom, a halogen atom, a nitro group or a cyano group,
Z is selected from H, pyridyl or optionally substituted phenyl, optionally substituted alkyl groups, alkoxy groups, halogens, nitro, cyano and carboxy groups,
W is selected from H, pyridyl or optionally substituted phenyl, optionally substituted alkyl groups, alkoxy groups, halogens, nitro, cyano and carboxy groups,
W¹ is H or optionally substituted alkyl, alkoxy or optionally substituted phenyl, and
Z¹ is H, or optionally substituted alkyl,
R³ and R⁴ are independently H, hydroxy, alkyl, alkoxy, aralkoxy, nitro, cyano or halogen,
D is an optionally substituted compound of the formula 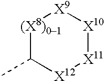
which can be saturated, or can have one, two or three double bonds, wherein
X⁸ to X¹¹ are independently CH, N. N⁺(R⁷), or CR⁸, if unsaturated, or
X⁸ to X¹¹ are independently CH₂, NH, NH⁺(R⁷), O. or S if saturated,
X¹² is CHA, NA, CH₂, NH, NH⁺(R⁷), or CHR⁸, if saturated or
X¹²is CA, NA⁺, CH, N, N⁺(R⁷), or CR⁸, if unsaturated and
A is H or an isoster of a carboxylic acid, PO₃R⁵R⁶, a tetrazole, an amide, an ester or an acid anhydride,
R⁶, R⁷ R⁸ and R⁹ are independently selected from H, optionally substituted C₁-C₉ branched or straight chain alkyl, or optionally substituted C₂-C₉ branched or straight chain alkenyl, or optionally substituted C₃-C₈ cycloalkyl, or an optionally substituted C₅-C₇ cycloalkenyl, or an optionally substituted aryl residue.

2. Compounds according to claim 1, wherein D has the following formula: 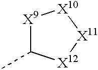
wherein the residues are as defined above.

3. Compounds according to claim 1, wherein D has the following formula: 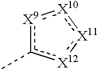
wherein the residues are as defined above.

4. Compounds according to claim 1, wherein D has the following formula: 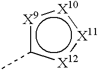
wherein the residues are as defined above.

5. Compounds according to claim 1, wherein D has the following formula: 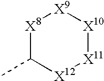
wherein the residues are as defined above.

6. Compounds according to claim 1, wherein D has the following formula: 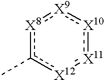
wherein the residues are as defined above.

7. Compounds according to claim 1, wherein D has the following formula: 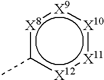
wherein the residues are as defined above.

8. Compounds according to any one of the preceding claims, wherein B has the following formula: 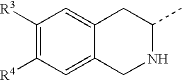
wherein the residues are as defined above.

9. Compounds according to any one of claims 1-7, wherein B has the following formula: 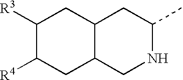
wherein the residues are as defined above.

10. A compound according to claim 1, selected from the group consisting of:
1-cyclopentyl-3-methyl-1-oxo-2-pentanaminium chloride,
1-cyclopentyl-3-methyl-1-oxo-2-butanaminium chloride,
1-cyclopentyl-3,3-dimethyl-1-oxo-2-butanaminium chloride,
1-cyclohexyl-3,3-dimethyl-1-oxo-2-butanaminium chloride,
3-(cyclopentylcarbonyl)-1,2,3,4-tetrahydroisoquinolinium chloride, and
N-(2-cyclopentyl-2-oxoethyl)cyclohexanaminium chloride.

11. A pharmaceutical composition for parenteral, enteral or oral administration, characterised in that it contains at least one compound according to any one of the preceding claims optionally in combination with customary carriers and/or excipients.

12. Use of compounds or pharmaceutical compositions according to any one of the preceding claims for the preparation of a medicament for the in vivo inhibition of DP IV and/or DP IV-like enzymes.

13. Use of compounds or pharmaceutical compositions according to any one of claims 1 to 11 for the preparation of a medicament for the treatment of diseases of mammals that can be treated by modulation of the DP IV activity of a mammal.

14. Use according to claim 12 or 13 for the treatment of metabolic diseases of humans.

15. The use according to claims 12, 13 or 14 for the treatment of impaired glucose tolerance, glucosuria, hyperlipidaemia, metabolic acidosis, diabetes mellitus, diabetic neuropathy or nephropathy or of sequelae caused by diabetes mellitus, neurodegenerative diseases or disturbance of signal action at the cells of the islets of Langerhans and insulin sensitivity in the peripheral tissue in the postprandial phase of mammals.

16. The use according to claims 12, 13 or 14 for the treatment of metabolism-related hypertension or cardiovascular sequelae caused by hypertension in mammals.

17. The use according to claims 12, 13 or 14 for the prophylaxis or treatment of skin diseases or diseases of the mucosae, autoimmune diseases or inflammatory conditions.

18. The use according to claims 12, 13 or 14 for the treatment of psychosomatic, neuro-psychiatric or depressive illnesses, such as anxiety, depression, sleep disorders, chronic fatigue, schizophrenia, epilepsy, nutritional disorders, spasm and chronic pain.

19. The use according to claims 12, 13 or 14 for the chronic treatment of chronic metabolic diseases in humans.

20. The use according to claims 12, 13 or 14 for the chronic treatment of chronically impaired glucose tolerance, chronic glucosuria, chronic hyperlipidaemia, chronic metabolic acidosis, chronic diabetes mellitus, chronic diabetic neuropathy or nephropathy or of chronic sequelae caused by diabetes mellitus, chronic neurodegenerative diseases or chronic disturbance of signal action at the cells of the islets of Langerhans or chronic insulin sensitivity in the peripheral tissue in the postprandial phase of mammals.

21. The use according to claims 12, 13 or 14 for the chronic treatment of metabolism-related hypertension or of chronic cardiovascular sequelae caused by chronic hypertension in mammals.

22. The use according to claims 12, 13 or 14 for the chronic treatment of chronic psychosomatic, chronic neuropsychiatric or depressive illnesses, such as chronic anxiety, chronic depression, chronic sleep disorders, chronic fatigue, chronic schizophrenia, chronic epilepsy, chronic nutritional disorders, spasm and chronic pain.
